(19) United States
(12) Patent Application Publication
(10) Pub. No.: US 2010/0298884 A1
(43) Pub. Date: Nov. 25, 2010

(54) Polyaxial Auxiliary Connector

(75) Inventors: Ahmad Faizan, Randolph, NJ (US); Mahmoud F. Abdelgany, Rockaway, NJ (US)

Correspondence Address: Rahman LLC;Mohammad Rahman, 10025 Governor Warfield Parkway, Suite 110, Columbia, MD, 21044, US

(73) Assignee: CUSTOM SPINE, INC., Parsippany, NJ (US)

(21) Appl. No.: 12/470,283

(22) Filed: May 21, 2009

(51) Int. Cl. A61B 17/70

(52) U.S. Cl. 606/266

(57) ABSTRACT

A polyaxial auxiliary connector assembly and method includes a first component comprising a base comprising at least one female socket; and a member connected to the base, wherein the member comprises at least one aperture. A second component is operatively connected to the base and comprises a seat portion; a pair of upright arms extending from the seat portion and defining a first channel therebetween; an outwardly protruding and expandable bulbous male end positioned adjacent to the seat portion and in the female socket of the base; and a second channel positioned through the seat portion and the male end, wherein the first and second channels each comprise a longitudinal axis that are positioned orthogonal to one another; a pin positioned through the second channel causing the bulbous male end of the second component to outwardly expand in the female socket; and a blocker mechanism engaged with the second component.

### BACKGROUND

1. Technical Field

The embodiments herein generally relate to a polyaxial connector component, and, more particularly, to a polyaxial auxiliary rod or bar connector component for extending a construct longitudinally or bridging two rods transversely.

2. Description of the Related Art

Pedicle screw and rod systems are a common method of treating various conditions in the spine. An existing spinal construct is often extended further to adjacent levels. In such a scenario, the spinal rods at adjacent levels are connected to build a continuous construct. Generally, conventional longitudinal rod to rod connectors do not use polyaxial connector systems.

### SUMMARY

In view of the foregoing, an embodiment herein provides a polyaxial auxiliary connector assembly comprising a first component comprising a base comprising at least one female socket; and a member connected to the base, wherein the member comprises at least one aperture. The assembly further comprises a second component operatively connected to the base, wherein the second component comprises a seat portion; a pair of upright arms extending from the seat portion and defining a first channel therebetween; an outwardly protruding and expandable bulbous male end positioned adjacent to the seat portion and in the female socket of the base; and a second channel positioned through the seat portion and the male end, wherein the first channel and the second channel each comprise a longitudinal axis that are positioned orthogonal to one another; a pin positioned through the second channel causing the bulbous male end of the second component to outwardly expand in the female socket; and a blocker mechanism engaged with the second component.

The member may outwardly extend from the base and is substantially parallel to the second component. The first component may be substantially L-shaped. The base may comprise a length defined along a first axis of the first component, wherein the member comprises a height defined along a second axis of the first component, and wherein the first axis and the second axis are orthogonal to one another. The at least one aperture of the member preferably comprises a first aperture comprising a longitudinal axis positioned substantially parallel to the second axis; and a second aperture comprising a longitudinal axis positioned orthogonal to each of the first axis and the second axis. Any of the first aperture and the second aperture may comprise grooves. The upper surface of the member may be planar with an upper surface of the base. The longitudinal axis of the first channel may be aligned and parallel to the longitudinal axis of the first aperture. The longitudinal axis of the first channel may be orthogonal to the longitudinal axis of the first aperture. Preferably, the first component is unanchored to bone.

Another embodiment provides a polyaxial auxiliary apparatus comprising at least two pedicle screw systems, wherein each pedicle screw system comprises a bone fixator component attached to bone; a first screw head attached to the bone fixator component, wherein the first screw head comprises a first channel; a longitudinal member positioned through the first channel; and a first blocker mechanism that retains the longitudinal member to the first screw head. The apparatus further comprises at least one connector component operatively connected to the at least two pedicle screw systems, wherein each connector component comprises a base comprising at least one female socket; and a member connected to the base, wherein the member comprises at least one aperture. The apparatus further comprises a second screw head operatively connected to the base, wherein the second screw head comprises a second channel that receives a portion of the longitudinal member; and a second blocker mechanism that retains the portion of the longitudinal member in the second screw head.

The second screw head may comprise a seat portion; a pair of upright arms extending from the seat portion and defining the second channel therebetween; an outwardly protruding and expandable bulbous male end positioned adjacent to the seat portion and in the female socket of the base; a third channel positioned through the seat portion and the male end, wherein the second channel and the third channel each comprise a longitudinal axis that are positioned orthogonal to one another; and a pin positioned through the third channel causing the bulbous male end of the second screw head to outwardly expand in the female socket; wherein the member outwardly extends from the base and is substantially parallel to the second screw head. The member may outwardly extend from the base and is substantially parallel to the first and second screw head. The base may comprise a length defined along a first axis of the connector component, wherein the member comprises a height defined along a second axis of the connector component, and wherein the first axis and the second axis are orthogonal to one another.

The at least one aperture of the member may comprise a first aperture comprising a longitudinal axis positioned substantially parallel to the second axis; and a second aperture comprising a longitudinal axis positioned orthogonal to each of the first axis and the second axis. Any of the first aperture and the second aperture may comprise grooves. The longitudinal axis of the second channel may be aligned and parallel to the longitudinal axis of the first aperture. The longitudinal axis of the second channel may be orthogonal to the longitudinal axis of the first aperture. The at least two pedicle screw systems may comprise a first pedicle screw system and a second pedicle screw system operatively connected to each other by the connector component, wherein the longitudinal member of the first pedicle screw system engages the at least one aperture of the connector component, and wherein the longitudinal member of the second pedicle screw system engages the second channel of the second screw head.

Another embodiment provides a method of connecting two longitudinal components of a plurality of pedicle screw assemblies, the method comprising providing a connector mechanism comprising a base component having at least one female socket; and at least one screw head having a receiving section and an outwardly protruding and expandable bulbous male end, wherein the female socket receives the bulbous male end, and wherein the base component comprises at least one aperture. The method further comprises adjoining a first pedicle screw assembly to a second pedicle screw assembly by connecting each of the assemblies to the connector mechanism.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A is a top perspective view of a polyaxial auxiliary connector mechanism according to an embodiment herein;

FIG. 1B is a first side view of the polyaxial auxiliary connector mechanism of FIG. 1A according to an embodiment herein;

FIG. 1C is a second side view of the polyaxial auxiliary connector mechanism of FIG. 1A according to an embodiment herein;

FIG. 2 is an exploded view of a polyaxial auxiliary connector assembly according to an embodiment herein;

FIG. 3 is a perspective view of a first component of the polyaxial auxiliary connector mechanism of FIG. 1A according to an embodiment herein;

FIG. 4A is a perspective view of a second component of the polyaxial auxiliary connector mechanism of FIG. 1A according to an embodiment herein;

FIG. 4B is a cross-sectional view of the second component of FIG. 4A according to an embodiment herein;

FIG. 5 is a perspective view of the blocker screw of the assembly of FIG. 2 according to an embodiment herein;

FIG. 6 is a perspective view of the set screw of the assembly of FIG. 2 according to an embodiment herein;

FIG. 7A is a perspective view of the saddle pin of the assembly of FIG. 2 according to an embodiment herein;

FIG. 7B is a side view of the stem portion of the saddle pin of the assembly of FIG. 2 according to an embodiment herein;

FIG. 8A is a perspective view of an apparatus according to a first embodiment herein;

FIG. 8B is a perspective view of an apparatus according to a second embodiment herein;

FIG. 9A is a perspective view of an alternative polyaxial auxiliary connector assembly according to an embodiment herein;

FIG. 9B is an exploded view of the polyaxial auxiliary connector assembly of FIG. 9A according to an embodiment herein; and

FIG. 10 is a flow diagram illustrating a preferred method according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

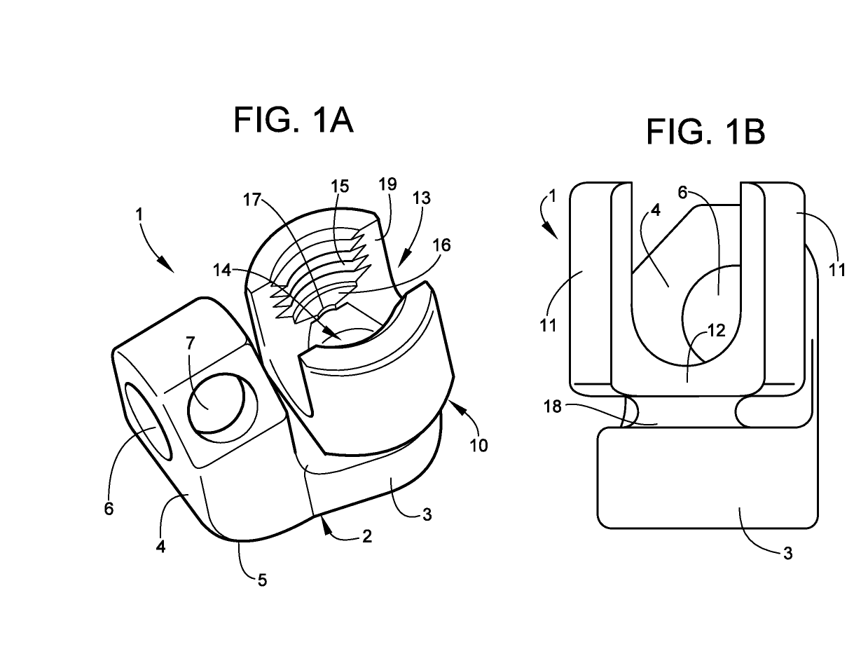

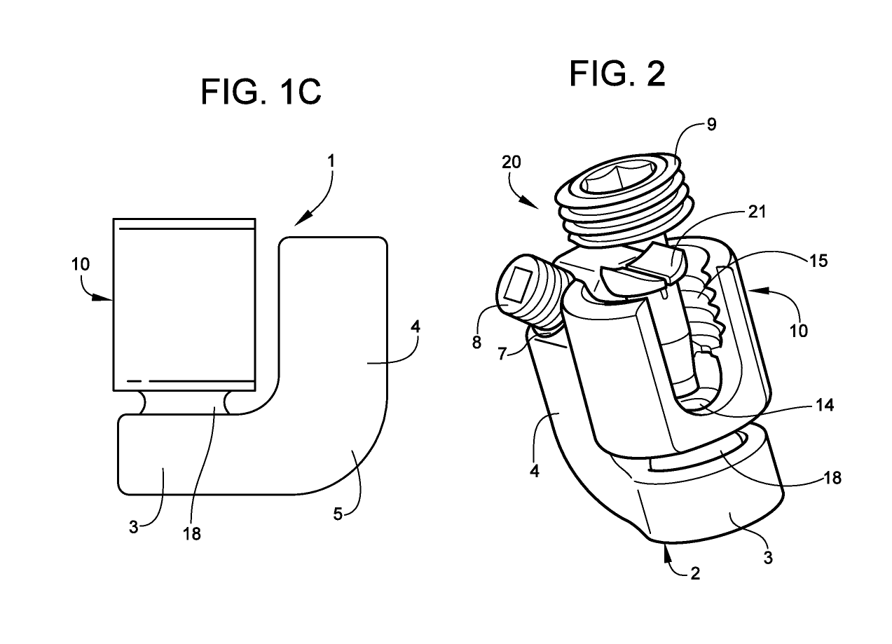

The embodiments herein provide a polyaxial auxiliary connector mechanism that may be used to connect multiple spinal rods longitudinally or transversely. Referring now to the drawings, and more particularly to FIGS. 1A through 10, where similar reference characters denote corresponding features consistently throughout the figures, there are shown preferred embodiments.

Referring to FIGS. 1A through 1C, a polyaxial auxiliary connector mechanism 1 includes a connector 2 that connects to a polyaxial screw head 10. The connector 2 comprises a base component 3 and an arm component 4. A generally curved joining section 5 connects the base component 3 with the arm component 4. The screw head 10 comprises an outwardly protruding and expandable bulbous male end 18 that connects with the base component 3, as further described below. The arm component 4 comprises a plurality of apertures 6, 7. Preferably, the apertures 6, 7 are positioned orthogonal to one another and may be configured with threads. The screw head 10 comprises a pair of upright parallel arms 11 that are joined by a seat 12 to form a substantially “U-shaped” configuration. An open receiving channel 13 is formed in between the upright arms 11. Moreover, each arm 11 may comprise threads 15 configured on an inner surface 19 of the arms 11 such that the inner surface 19 (and threads 15) face the open receiving channel 13. Positioned below the threads 15 is a locking edge 16 comprising a notch 17 substantially centrally located therein. A channel 14 is disposed substantially centrally through the seat 12 and through the bulbous male end 18 of the screw head 10, wherein the channel 14 is oriented substantially transverse to the orientation of receiving channel 13.

FIG. 2, with reference to FIGS. 1A through 1C, illustrates an exploded view of a polyaxial auxiliary connector assembly 20 according to an embodiment herein. The assembly 47 comprises the polyaxial auxiliary connector mechanism 1 of FIGS. 1A through 1C as well as a set screw 8, blocker screw 9, and saddle pin 21. The set screw 8 is configured to engage aperture 7, the blocker screw 9 is configured to engage threads 15 of screw head 10, and saddle pin 21 is configured to be seated within channel 14 of the screw head 10.

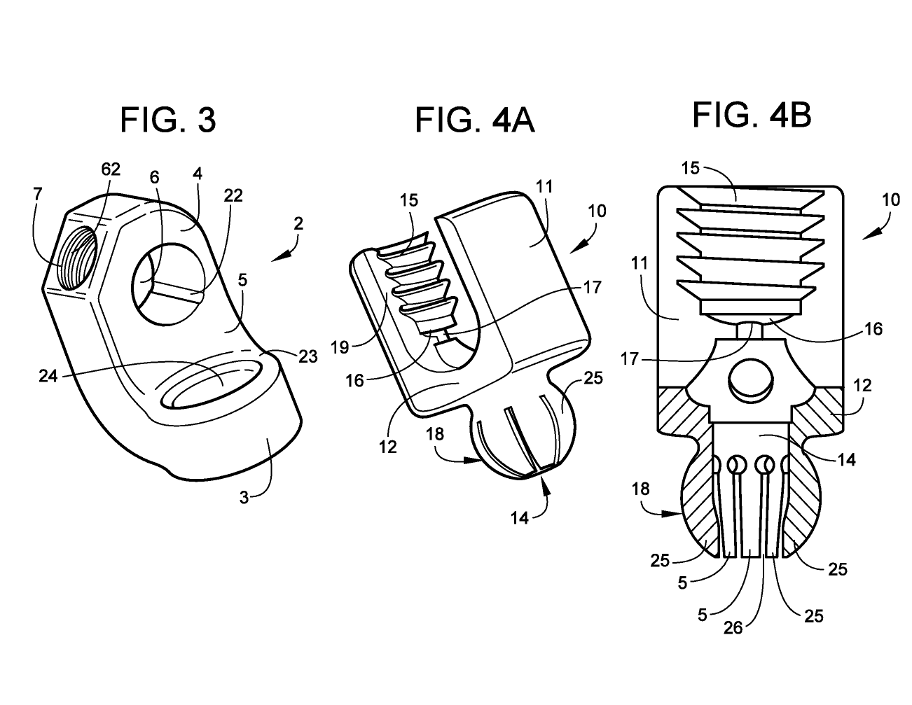

FIG. 3, with reference to FIGS. 1A through 2, is a perspective view of the connector 2 according to an embodiment herein. In this embodiment, the connector 2 is substantially “L-shaped” with the arm component 4 extending upright from one side of the base component 3 where the joining section 5 is positioned. The aperture 7 in FIG. 3 is shown having threads 62 to receive the threaded set screw 8. However, those skilled in the art would understand that aperture 7 may be configured with or without threads 62, and if configured without threads 62, then the set screw 8 may be configured as a push-pin type set screw/pin. Aperture 6 may be configured with one or more longitudinal notch(es) 22, which may extend the entire length of the aperture 6. However, those skilled in the art would understand that aperture 6 may be configured with or without notch(es) 22. The base component 3 of the connector 2 comprises a top portion 23 comprising a socket 24 configured therein. Preferably, the socket 24 is configured as a substantially concave or semi-spherical shaped socket 24 that is dimensioned to receive the bulbous male end 18 of the screw head 10 such that the male end 18 is permitted to rotate in socket 24 until the male end is locked into position.

FIGS. 4A and 4B, with reference to FIGS. 1A through 3, illustrate isolated views of the screw head 10 according to an embodiment herein. The male end 18 of the screw head 10 further comprises a plurality of bendable flanges 25 separated by slots 26 such that the flanges 25 outwardly expand when the saddle pin 21 is inserted through channel 14 and pushes against flanges 25 thereby causing them to outwardly bend. During use, a surgeon would position the male end 18 of the screw head 10 into the socket 24 of the connector 2, and upon attaining the proper angle of the screw head 10 would insert the saddle pin 21 into the channel 14 causing the male end 18 to outwardly expand and lock into the socket 24.

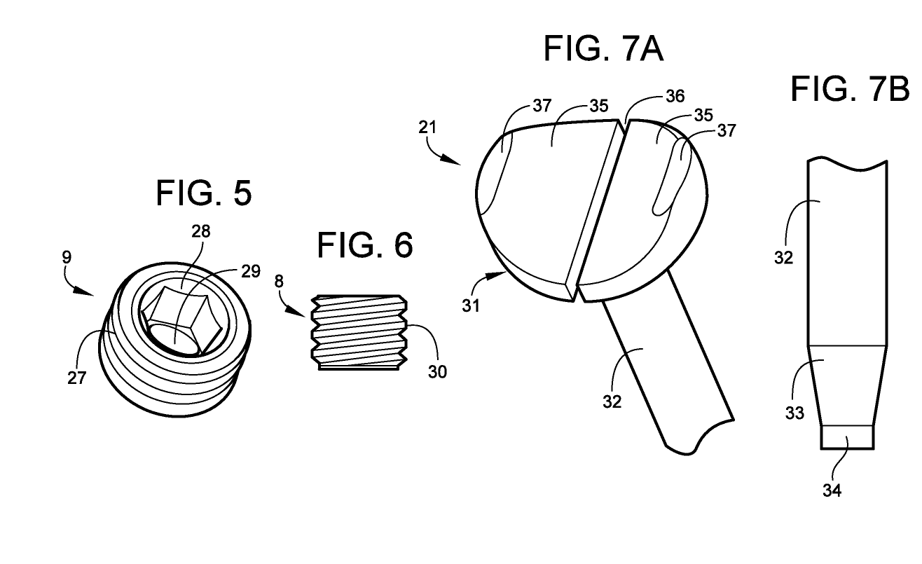

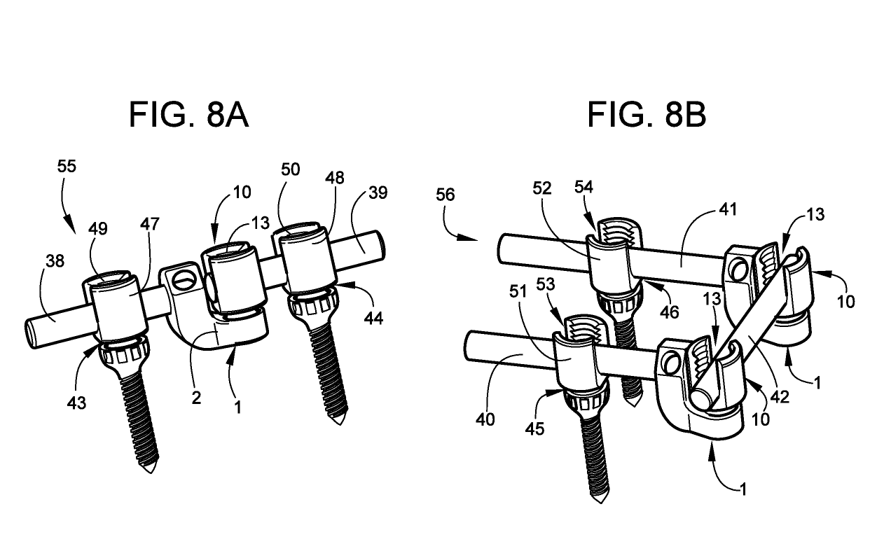

FIG. 5, with reference to FIGS. 1A through 4B, is a perspective view of the blocker screw 9 according to an embodiment herein. The blocker screw 9 includes a standard buttress thread 27 configured along an outer perimeter thereon. The blocker screw 9 helps to secure a longitudinal member (i.e., longitudinal members 38-42 in FIGS. 8A and 8B) inside the screw head 10. The threads 27 of the blocker screw 9 are configured to engage the complimentary threads 15 of the screw head 10. Additionally, the blocker screw 9 aids in preventing the expansion of the screw head 10 when torqued on the longitudinal member (i.e., longitudinal members 38-42 in FIGS. 8A and 8B), directing the counterforce more vertically than horizontally. The top/bottom 28 of the blocker screw 9 has a fastening feature 29 such as a hex or square lock feature or any other suitable configuration to allow high torque to be applied to the blocker screw 9. Moreover, the blocker screw 9 may have a “timed” thread 27 that is consistently and precisely related to the blocker driving tool (not shown) to help calculate the torsional and vertical position of the blocker screw 9 thereby assisting the torque measurement applied to the blocker screw 9. Additionally, those skilled in the art would understand that a blocker screw may be configured in a non-threaded configuration such that it would function as a push-pin type of mechanism to engage the screw head 10, which in this embodiment, would not include threads 15.

FIG. 6, with reference to FIGS. 1A through 5, is a perspective view of the set screw 8 according to an embodiment herein. The set screw 8 engages aperture 7 of the connector 2. When embodied with threads 30, the set screw 8 engages the complementary threads 62 of aperture 7. Again, those skilled in the art would understand that aperture 7 may be configured with or without threads 62, and if configured without threads 62, then the set screw 8 may be configured as a push-pin type set screw/pin (i.e., without threads 30).

FIGS. 7A and 7B, with reference to FIGS. 1A through 6, are isolated views of the saddle pin 21 according to an embodiment herein. The saddle pin 21 comprises a head portion 31 and a stem portion 32 extending therefrom. In one embodiment, the stem portion 32 may comprise a tapered configuration comprising tapered section 33 and generally cylindrical end section 34. However, in other embodiments, the stem portion 32 may be uniformly configured in a cylindrical configuration or in a gradually tapered configuration without a cylindrical end section 34. In one embodiment, the material of the stem portion 32 of the saddle pin 21 comprises ceramic and has a higher hardness and compressive yield strength than the comparative hardness and compressive yield strength of Ti₆Al₄V, which is the material which may be used in constructing the screw head 10.

The head portion 31 of the saddle pin 21 provides a proper seat for the longitudinal member (i.e., longitudinal members 38-42 in FIGS. 8A and 8B) and avoids notching a typical titanium longitudinal member (titanium is very notch sensitive). Furthermore, the saddle pin 21 allows one to accommodate multiple sizes of longitudinal members (i.e., longitudinal members 38-42 in FIGS. 8A and 8B) in the same screw head 10. In one embodiment, the head portion 31 includes a pair of seats 35 separated by slot 36, which is positioned at a substantial midpoint of the head portion 31. A secondary locking mechanism 37 may be configured on ends of the seats 35 to further achieve locking of the saddle pin 21 once it is inserted in the screw head 10 such that each locking mechanism 37 engages the complementary notch 17 of the locking edge 16 configured on the inner surface 19 of the screw head 10. In another embodiment, the saddle pin 21 may be configured without secondary locking mechanism 37. The saddle pin 21 is configured with slot 36 through the center to allow expansion/contraction of the seats 35 of the head portion 31 with respect to one another. When positioned in the channel 14 of the screw head 10, the saddle pin 21 expands the male bulbous end 18 of the screw head 10 into the female spherical socket 24 in the connector 2 causing the screw head 10 to lock (or start locking).

FIGS. 8A and 8B, with respect to FIGS. 1A through 7B, are perspective views of an apparatus 55, 56, respectively, according to the embodiments herein. In apparatus 55 shown in FIG. 8A, the polyaxial auxiliary connector mechanism 1 serves as a linear connector for two pedicle screw assemblies 43, 44, whereby the longitudinal member 39 of pedicle screw assembly 44 connects with the screw head 10 (i.e., through receiving channel 13) of mechanism 1 while the longitudinal member 38 of pedicle screw assembly 43 connects with the connector 2 (i.e., through aperture 6). In this embodiment, the receiving channel 13 of screw head 10 is aligned with the corresponding receiving channels 49, 50 of the corresponding screw heads 47, 48 of pedicle screw assemblies 43, 44, respectively.

In apparatus 56 shown in FIG. 8B, a pair of polyaxial auxiliary connector mechanisms 1 connect a pair of pedicle screw assemblies 45, 46 such that the pedicle screw assemblies 45, 46 are positioned substantially parallel to one another. In this embodiment, the receiving channels 13 of the screw heads 10 of the mechanism 1 are positioned substantially perpendicular to the channels 53, 54 of the corresponding screw heads 51, 52 of the pedicle screw assemblies 45, 46, respectively. Accordingly, the longitudinal members 40, 41 of pedicle screw assemblies 45, 46, respectively, connect in aperture 6 of each connector mechanism 1, and longitudinal member 42 is positioned in receiving channel 13 of each screw head 10 to facilitate the cross-linking feature of apparatus 56. Pedicle screw assemblies 43-46 may be configured similar to any appropriate pedicle screw assembly known in the art.

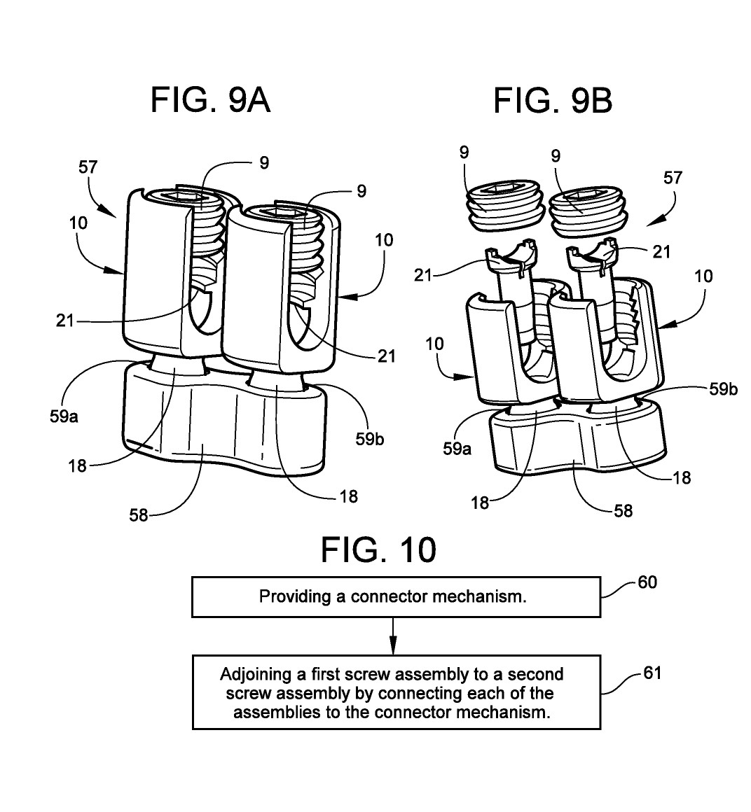

FIGS. 9A and 9B, with reference to FIGS. 1A through 8B, illustrate an alternative polyaxial auxiliary connector assembly 57 according to an embodiment herein. In this embodiment, the connector 58 is configured with a plurality of female sockets 59a, 59b, which allow a plurality of screw heads 10 to be seated in one connector 58. The features of the screw head 10, blocker screw 9, and saddle pin 21 are consistent with this embodiment as with the embodiment shown and described in FIG. 2.

FIG. 10, with reference to FIGS. 1A through 9B, is a flow diagram illustrating a method of connecting two longitudinal components (i.e., longitudinal members 38, 39 or longitudinal members 40, 41) of a plurality of pedicle screw assemblies (i.e., polyaxial pedicle screw assemblies 43, 44 or polyaxial pedicle screw assemblies 45, 46) according to an embodiment herein. The method comprises providing (60) a connector mechanism 1, 57 comprising a base component 2, 58 having at least one female socket 24, 59a, 59b; and at least one screw head 10 having a receiving section 13 and an outwardly protruding and expandable bulbous male end 18, wherein the female socket 24, 59a, 59b receives the bulbous male end 18, and wherein the base component 2, 58 comprises at least one aperture 6, 7. The method further comprises adjoining (61) a first pedicle screw assembly (i.e., polyaxial pedicle screw assembly 43, 45) to a second pedicle screw assembly (i.e., polyaxial pedicle screw assemblies 44, 46) by connecting each of the assemblies (i.e., polyaxial pedicle screw assemblies 43, 44 or polyaxial pedicle screw assemblies 45, 46) to the connector mechanism 1, 57. Generally, the embodiments herein provide a polyaxial auxiliary rod/bar connector assembly 20, 57 used in the spine for extending/connecting pedicle screw assemblies 43, 44 longitudinally or bridging a plurality of pedicle screw assemblies 45, 46 transversely.

In assembling the embodiments herein, the screw head 10 is preassembled in the base component 3. Similarly, the saddle pin 21 is preloaded in the screw head 10. During surgery, the surgeon tightens the blocker screw 9 which applies a downward force on the saddle pin 21 and thereby locks the screw head 10 in place.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A polyaxial auxiliary connector assembly comprising:
a first component comprising:
a base comprising at least one female socket; and
a member connected to said base, wherein said member comprises at least one aperture;
a second component operatively connected to said base, wherein said second component comprises:
a seat portion;
a pair of upright arms extending from said seat portion and defining a first channel therebetween;
an outwardly protruding and expandable bulbous male end positioned adjacent to said seat portion and in said female socket of said base; and
a second channel positioned through said seat portion and said male end, wherein said first channel and said second channel each comprise a longitudinal axis that are positioned orthogonal to one another;
a pin positioned through said second channel causing said bulbous male end of said second component to outwardly expand in said female socket; and
a blocker mechanism engaged with said second component.

2. The assembly of claim 1, wherein said member outwardly extends from said base and is substantially parallel to said second component.

3. The assembly of claim 2, wherein said first component is substantially L-shaped.

4. The assembly of claim 1, wherein said base comprises a length defined along a first axis of said first component, wherein said member comprises a height defined along a second axis of said first component, and wherein said first axis and said second axis are orthogonal to one another.

5. The assembly of claim 4, wherein said at least one aperture of said member comprises:
a first aperture comprising a longitudinal axis positioned substantially parallel to said second axis; and
a second aperture comprising a longitudinal axis positioned orthogonal to each of said first axis and said second axis.

6. The assembly of claim 5, wherein any of said first aperture and said second aperture comprise grooves.

7. The assembly of claim 1, wherein an upper surface of said member is planar with an upper surface of said base.

8. The assembly of claim 5, wherein said longitudinal axis of said first channel is aligned and parallel to said longitudinal axis of said first aperture.

9. The assembly of claim 5, wherein said longitudinal axis of said first channel is orthogonal to said longitudinal axis of said first aperture.

10. The assembly of claim 1, wherein said first component is unanchored to bone.

11. A polyaxial auxiliary apparatus comprising:
at least two pedicle screw systems, wherein each pedicle screw system comprises:
a bone fixator component attached to bone;
a first screw head attached to said bone fixator component, wherein said first screw head comprises a first channel;
a longitudinal member positioned through said first channel; and
a first blocker mechanism that retains said longitudinal member to said first screw head;
at least one connector component operatively connected to said at least two pedicle screw systems, wherein each connector component comprises:
a base comprising at least one female socket; and
a member connected to said base, wherein said member comprises at least one aperture;
a second screw head operatively connected to said base, wherein said second screw head comprises a second channel that receives a portion of said longitudinal member; and
a second blocker mechanism that retains said portion of said longitudinal member in said second screw head.

12. The apparatus of claim 11, wherein said second screw head comprises:
a seat portion;
a pair of upright arms extending from said seat portion and defining said second channel therebetween;
an outwardly protruding and expandable bulbous male end positioned adjacent to said seat portion and in said female socket of said base;
a third channel positioned through said seat portion and said male end, wherein said second channel and said third channel each comprise a longitudinal axis that are positioned orthogonal to one another; and
a pin positioned through said third channel causing said bulbous male end of said second screw head to outwardly expand in said female socket; wherein said member outwardly extends from said base and is substantially parallel to said second screw head.

13. The apparatus of claim 12, wherein said member outwardly extends from said base and is substantially parallel to said first and second screw head.

14. The apparatus of claim 11, wherein said base comprises a length defined along a first axis of said connector component, wherein said member comprises a height defined along a second axis of said connector component, and wherein said first axis and said second axis are orthogonal to one another.

15. The apparatus of claim 14, wherein said at least one aperture of said member comprises:
a first aperture comprising a longitudinal axis positioned substantially parallel to said second axis; and
a second aperture comprising a longitudinal axis positioned orthogonal to each of said first axis and said second axis.

16. The apparatus of claim 15, wherein any of said first aperture and said second aperture comprise grooves.

17. The apparatus of claim 15, wherein said longitudinal axis of said second channel is aligned and parallel to said longitudinal axis of said first aperture.

18. The apparatus of claim 15, wherein said longitudinal axis of said second channel is orthogonal to said longitudinal axis of said first aperture.

19. The apparatus of claim 11, wherein said at least two pedicle screw systems comprise a first pedicle screw system and a second pedicle screw system operatively connected to each other by said connector component, wherein the longitudinal member of said first pedicle screw system engages said at least one aperture of said connector component, and wherein the longitudinal member of said second pedicle screw system engages said second channel of said second screw head.

20. A method of connecting two longitudinal components of a plurality of pedicle screw assemblies, said method comprising:
providing a connector mechanism comprising:
a base component having at least one female socket; and
at least one screw head having a receiving section and an outwardly protruding and expandable bulbous male end,
wherein said female socket receives said bulbous male end, and
wherein said base component comprises at least one aperture;
adjoining a first pedicle screw assembly to a second pedicle screw assembly by connecting each of the assemblies to said connector mechanism.
